(19) United States
(12) Patent Application Publication
(10) Pub. No.: US 2021/0077787 A1
(43) Pub. Date: Mar. 18, 2021

(54) Magnetic Coupling to Prevent Guidewire Loss During Catheterization

(71) Applicant: Aaron Ginster, Fox Point, WI (US)

(72) Inventor: Aaron Ginster, Fox Point, WI (US)

(21) Appl. No.: 17/009,899

(22) Filed: Sep. 2, 2020

Related U.S. Application Data
(60) Provisional application No. 62/900,482, filed on Sep. 14, 2019.

(51) Int. Cl. A61M 25/09; A61M 25/00; A61M 25/01

(52) U.S. Cl. CPC A61M 25/09; A61M 25/0127; A61M 25/0097

(57) ABSTRACT

A guidewire comprising a magnetic element that would help to avoid accidental release of the guidewire. The magnetic element is located at a proximal one-fourth segment of the guidewire. The guidewire could be used in a variety of contexts, such as central venous catheterization, endovascular radiology procedures, or cardiac catheterization. Also disclosed are vascular instruments comprising a magnetic element or magnetically responsive element. Examples of such vascular instruments include finder needles, dilation cannulas, and various types of catheters. The vascular instrument could be used in combination with a magnetic guidewire to help to avoid accidental release of the guidewire.

### TECHNICAL FIELD

This invention relates to guidewires and intravascular catheters used in medical practice.

### BACKGROUND

A central venous catheter (also known as a central line) placed into a large vein is commonly used for administering medication or fluids. Central venous catheters are useful in various situations such as when peripheral venous access is impossible, large volume fluid resuscitation or vasopressors is needed, or long-term intravenous antibiotics or chemotherapy is being administered. The most common approach for performing this procedure is the Seldinger technique (or modified versions), in which the central line catheter is inserted via an access route created by a hollow bore needle in conjunction with a guidewire. The Seldinger technique is also used in interventional cardiac procedures and endovascular interventional radiology procedures.

FIGS. 1A-1F show an example of how a central venous catheterization procedure is conventionally performed. FIG. 1A shows a finder needle assembly 10, which comprises a syringe 12 and an introducer needle 14. The introducer needle 14 is punctured through the skin 16 and inserted into the target vein 18 until blood is aspirated into the syringe 12. As shown in FIG. 1B, the syringe 12 is then separated from the introducer needle 14 and removed. The next step uses a guidewire 20 that is held within a coiled guidewire sheath 22. The distal end of the guidewire 20 is inserted into the hub of the introducer needle 14.

The guidewire 20 is unwound out of the coiled sheath 22 as it is advanced in the direction of arrow 26 through the introducer needle 14 and into the target vein 18. As shown in FIG. 1C, when the guidewire 20 is inside the vein 18, a scalpel tip is used to make a small stab incision in the skin 16 against the guidewire 20 to make a slit just large enough to accommodate the dilator 30 (and eventually, the central venous catheter 40, as shown in FIGS. 1D and 1E).

FIG. 1D shows a dilator 30 that is used to widen the initial puncture site. The guidewire 20 is inserted into the dilator 30 at the opening at its distal tip and is threaded through the dilator 30 until the guidewire 20 exits out the opening on the other end of the dilator 30. While firmly grasping the guidewire 20 at its proximal end, the dilator 30 is pushed forward in the direction of arrow 32 through the skin 16 and subcutaneous tunnel as it tracks along the guidewire 20. Typically, twisting motion of the dilator 30 facilitates passage. This enlarges the puncture site and the tunnel passage into the target vein 18. When this task is finished, the dilator 30 is withdrawn backwards and removed off the guidewire 20.

FIG. 1E shows a single-lumen central venous catheter 40. For insertion of the catheter 40, the guidewire 20 is inserted into the opening at the distal tip of the catheter 40 and is threaded through the catheter 40 until the guidewire 20 exits out of the opening at the port 42. At the same time, while firmly holding the guidewire 20, the catheter 40 is advanced forward over guidewire 20 and pushed through the skin 16 and into the target vein 18. When the catheter 40 is securely in place, the guidewire 20 is pulled back in the direction of arrow 44 and removed entirely. FIG. 1F shows the catheter 40 positioned inside the target vein 18. Via catheter port 42, the catheter 40 is flushed with saline and secured to the skin by suture or tape.

This procedure is generally considered safe, but as with any medical procedure, there are numerous possible complications that could result. One complication that could occur is when the guidewire that is used in the Seldinger technique is inadvertently lost and released into the blood vessel or retained within the catheter. Factors that can lead to a missing or misplaced guidewire include inattention, operator inexperience, inadequate supervision of trainees, and overtired personnel.

If this mistake is recognized quickly, then the guidewire might still be trapped within the catheter and removed by clamping the catheter and guidewire together, and then taking out the catheter/guidewire en-bloc. If the guidewire has passed out of the catheter entirely and lost into the blood vessel, then an endovascular interventional procedure under radiologic guidance or surgery would have to be performed to retrieve the lost guidewire.

### SUMMARY

My invention uses magnetic coupling to address the problem of guidewire loss during a medical procedure. Magnetic coupling may be between two permanent magnets or between a permanent magnet and a magnetically responsive element. As used herein, “magnetic element” means a component that comprises a permanent magnetic material. As used herein, the term “magnetically responsive element” means a component that comprises a material that is not a permanent magnet, but is attracted to a magnetic field produced by a permanent magnet. Example of such materials include iron, nickel, cobalt, and alloys made therefrom such as stainless steel.

The magnetic element or magnetically responsive element may have any suitable shape or configuration, including strips, rods, cuffs, rings, wires, coils, cylindrical bands, etc., and have any suitable dimension (e.g. length, width, diameter, etc.) depending on the device or part thereof where the magnetic element or magnetically responsive element is located. The magnetic element or magnetically responsive element may be located at any suitable place on the relevant device. The magnetic element or magnetically responsive element may be a separate component or integrated with the relevant part of the device as a single unitary structure. For example, the magnetic element or magnetically responsive element may be embedded within a part of the device or joined to an exterior part of the device.

In one aspect, my invention is a magnetic guidewire that comprises a magnetic element. The guidewire is a wire-like structure that comprises a proximal end and a distal end. The guidewire further comprises a magnetic element located at a proximal one-fourth segment of the guidewire. In some embodiments, the magnetic element is located at a position within 5 cm from the proximal tip of the guidewire. In some embodiments, the guidewire does not have a magnetic element at a distal one-fourth segment of the guidewire. In some embodiments, the guidewire does not have a magnetic element on any other segment of the guidewire.

The guidewire may have any suitable length and diameter, depending on its intended site of use, patient body size, type of corresponding catheter, etc. For example, the guidewire may have a length of 2.5 to 600 cm long and a diameter of 0.6 to 3.3 mm wide. Longer guidewires are typically used in interventional cardiology or interventional radiology procedures, whereas shorter guidewires are used in central venous catheterization procedures.

Guidewires can have any of a variety of different configurations and material compositions. Many guidewires are made of metal such as stainless steel or a nitinol core wire wrapped in a stainless steel coil or braiding. Coiled or braided wire configurations improve flexibility, pushability, and kink resistance. Some guidewires have a floppy distal tip for easier navigation, while having a stiff body to enable good pushability. Some guidewires have a distal J-curve tip or pig tail configuration. Some guidewires are coated with a polymer, such as silicone or polytetrafluoroethylene (PTFE), to increase lubricity. Hydrophilic coatings are sometimes used to reduce friction during deployment and for easier navigation in tortuous vessels.

In another aspect, my invention is a vascular instrument designed to allow magnetic coupling with a guidewire to help in retaining the guidewire and avoid accidental release of the guidewire out of the instrument. In one embodiment, the vascular instrument comprises a magnetic element. The guidewire used with this vascular instrument may be a conventional guidewire, or may be a magnetic guidewire of my invention. For example, a conventional stainless steel guidewire could engage in magnetic coupling with the vascular instrument of my invention.

As used herein, “vascular instrument” means an elongated instrument with a hollow channel that is at least partially inserted into the patient's blood vessel. The blood vessel may be an artery or vein, such as external jugular vein, subclavian vein, femoral vein, radial artery, cephalic artery, femoral artery, coronary artery, carotid artery, cerebral artery, etc. Examples of vascular instruments include finder needles, dilation cannulas, infusion catheters (e.g. single lumen venous catheters, triple lumen venous catheters, arterial catheters, etc.), dialysis catheters, angioplasty catheters, peripherally inserted central catheters (PICC), snare catheters, aspiration catheters, guide catheters, balloon catheters, stent catheters, etc. The vascular instrument could be in-dwelling or temporary.

The vascular instrument may have any suitable length and diameter, depending on the type of instrument, its intended site of use, etc. For example, the vascular instrument could have a length in the range of 2.5 to 600 cm. The vascular instrument could have a diameter in the range of 3 to 18 French (1 to 6 mm).

The magnetic element for the vascular instrument may be positioned anywhere along the length of the instrument. In some embodiments, the magnetic element is located on a proximal one-fourth section of the vascular instrument. In some embodiments, the magnetic element is located at a position within 5 cm from the proximal tip of the vascular instrument. In some embodiments, the vascular instrument comprises an elongated tube with a hollow channel and the magnetic element is located on a proximal one-fourth section of the tube. In some embodiments, the magnetic element is located at a position within 5 cm from the proximal tip of the tube. The tube may be a single unitary structure or a complex of multiple structures (e.g. in a triple lumen catheter).

In another embodiment, the vascular instrument of my invention comprising a magnetically responsive element (e.g. made of stainless steel). The magnetically responsive element for the vascular instrument may be positioned anywhere along the length of the instrument. In some embodiments, the magnetically responsive element is located on a proximal one-fourth section of the vascular instrument. In some embodiments, the magnetically responsive element is located at a position within 5 cm from the proximal tip of the vascular instrument. In some embodiments, the vascular instrument comprises an elongated tube with a hollow channel and the magnetically responsive element is located on a proximal one-fourth section of the tube. In some embodiments, the magnetically responsive element is located at a position within 5 cm from the proximal tip of the tube. The tube may be a single unitary structure or a complex of multiple structures (e.g. in a triple lumen catheter).

My invention also contemplates kits that comprise one or more of my invention elements above. In one embodiment, my invention is a vascular catheterization kit comprising a magnetic guidewire of my invention. The kit may further include one or more other conventional components, such as introducer sheaths, catheters, syringes, dilators, etc. In some embodiments, the kit further comprises a dilation cannula (vascular instrument) of my invention. In some embodiments, the kit further comprises an infusion catheter (vascular instrument) of my invention. In another embodiment, my invention is a vascular catheterization kit comprising a vascular instrument of my invention. The kit may further include one or more other conventional components, such as introducer sheaths, catheters, syringes, dilators, etc. In some embodiments, the kit further comprises a magnetic guidewire of my invention.

In another aspect, my invention is a method of retrieving a guidewire. The guidewire may be lost or misplaced (e.g. trapped within a vascular instrument or inside the patient's body). In one embodiment, the method is for retrieving a magnetic guidewire of my invention. The magnetic guidewire is lost inside the patient's body (e.g. inside a blood vessel or in the heart). The method comprises inserting an endovascular retrieval instrument (e.g. snare catheter) into the patient's body and guiding it towards the lost guidewire. The method further comprises magnetically coupling the magnetic guidewire with the endovascular retrieval instrument. The magnetic guidewire could be further ensnared by the endovascular retrieval instrument. The endovascular retrieval instrument is withdrawn from the patient's body with the magnetic guidewire in tow.

In another embodiment, the method is for retrieving a (first) magnetic guidewire of my invention using a second magnetic guidewire. The first magnetic guidewire is trapped inside a vascular instrument (e.g. infusion catheter). The second magnetic guidewire is inserted into the vascular instrument at its proximal opening with the guidewire in a proximal end-first (reverse) orientation. When the second magnetic guidewire meets the first magnetic guidewire inside the vascular instrument, the first magnetic guidewire is magnetically coupled to the second magnetic guidewire. The second magnetic guidewire is withdrawn from the vascular instrument with the first magnetic guidewire in tow.

In another aspect, my invention is a method of vascular catheterization using a magnetic guidewire of my invention with a vascular instrument of my invention. In some embodiments, the method comprises inserting the vascular instrument (e.g. finder needle) into a blood vessel. The distal tip of the guidewire is inserted into a proximal opening of the vascular instrument. The guidewire is advanced through the vascular instrument and into the blood vessel. If the guidewire travels too far in a distal direction such that the magnetic element on the guidewire meets the magnetic element or magnetically responsive element on the vascular instrument, magnetic coupling between the two impedes further distal travel of the guidewire down the vascular instrument.

In some embodiments, the method comprises having the guidewire placed inside a patient's blood vessel. The proximal tip of the guidewire is inserted into a distal opening of the vascular instrument. The vascular instrument is advanced over the guidewire into the blood vessel such that the guidewire travels in a proximal direction relative to the vascular instrument. However, the guidewire could also travel in a distal direction (i.e. downward) relative to the vascular instrument. For example, the operator may inadvertently fail to keep a firm grip on the guidewire during the procedure. If the guidewire travels too far in a distal direction such that the magnetic element on the guidewire meets the magnetic element or magnetically responsive element on the vascular instrument, magnetic coupling between the two impedes further distal travel of the guidewire down the vascular instrument.

In some embodiments, the magnetic guidewire has a magnetic element oriented with its first polarity on a proximal side and its second opposite polarity on a distal side. The vascular instrument has a magnetic element oriented in the reverse direction, i.e. with its first polarity on a distal side and its second opposite polarity on a proximal side. During insertion of the vascular instrument into the patient over the magnetic guidewire, the vascular instrument is pushed against the repulsive resistance when the first polarity on the distal side for the magnetic element on the vascular instrument meets the same first polarity on the proximal side for the magnetic element on the guidewire. The two magnetic elements traverse each other as the vascular instrument is advanced over the guidewire. If the guidewire should travel backwards (i.e. distal direction) within the vascular instrument, the guidewire meets repulsive resistance when the second polarity on the proximal side for the magnetic element on the vascular instrument meets the same second polarity on the distal side for the magnetic element on the guidewire. This repulsive resistance helps to avoid accidental loss of the guidewire.

### BRIEF DESCRIPTION OF THE DRAWINGS

FIGS. 1A-1F show an example of how a central venous catheterization procedure is conventionally performed.

FIG. 2 demonstrates the partitioning of a guidewire into four equal one-fourth segments.

FIG. 3 shows an example of a guidewire of my invention.

FIG. 4 shows an example of a dilation cannula of my invention used in combination with the guidewire shown in FIG. 3.

FIG. 5 shows an example central venous catheter of my invention.

FIGS. 6A and 6B (on the landscape oriented drawing sheet alongside FIG. 8 below) show how the polarity of the magnetic elements could be arranged for enhancing function.

FIGS. 7A-7C illustrate an example of a lost guidewire situation.

FIG. 8 illustrate an example of how two magnetic guidewires of my invention could be used in a lost guidewire situation.

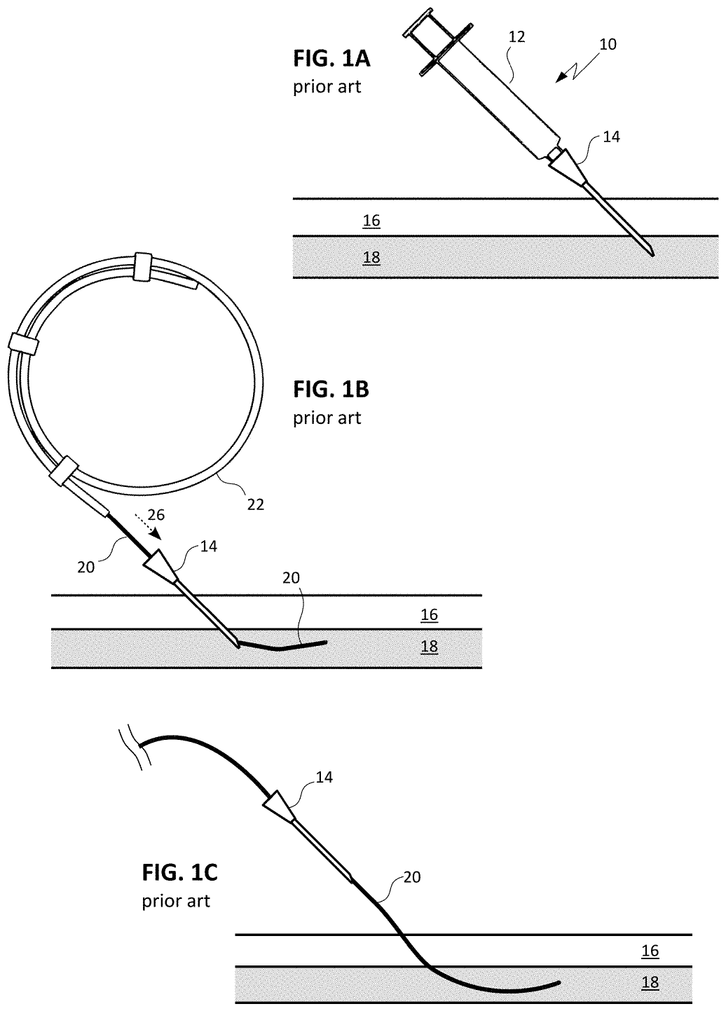

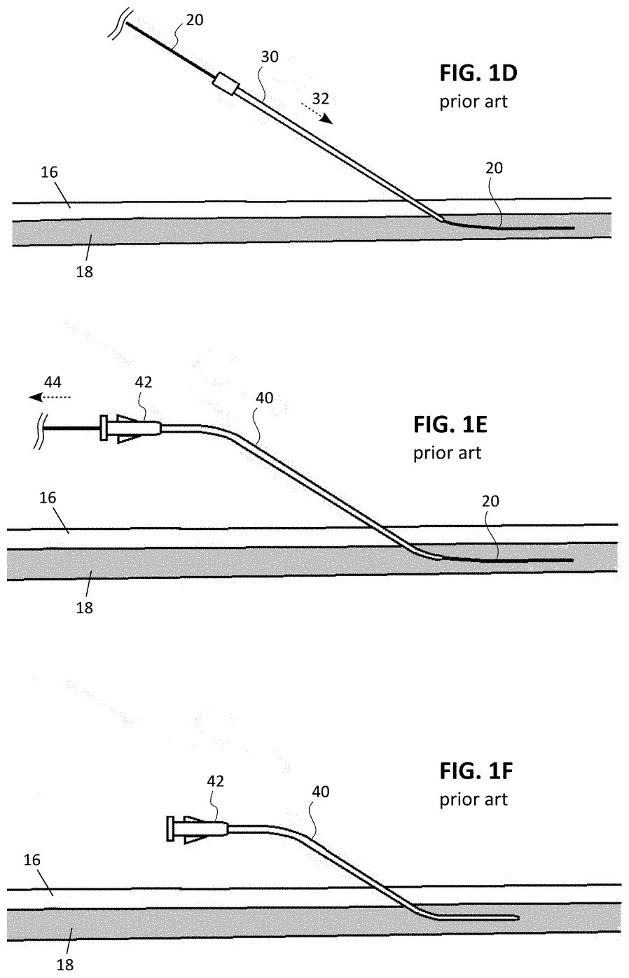

### DETAILED DESCRIPTION

To assist in understanding my invention, reference is made to the accompanying drawings to show by way of illustration specific embodiments in which the invention may be practiced.

### 1. GUIDEWIRE

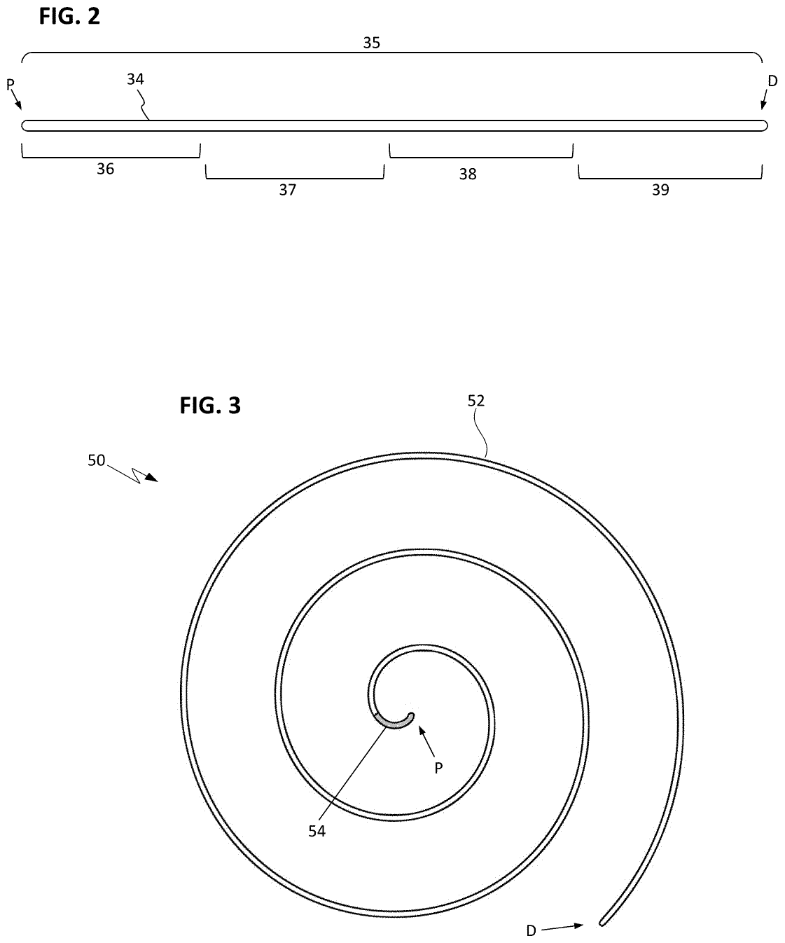

In one aspect, my invention is a guidewire that comprises a magnetic element to allow magnetic coupling with the relevant device or instrument to help in retaining the guidewire and avoid accidental release of the guidewire out of the instrument. The magnetic element is located somewhere on the proximal one-fourth segment of the guidewire. This is demonstrated in FIG. 2, which depicts a guidewire 34 in a straightened configuration. To specify orientation, the distal end is indicated by the reference label “D” and the proximal end is indicated by the reference label “P”. There are a variety of possible ways to distinguish the proximal end of a guidewire from its distal end. That is, the guidewire could be longitudinally asymmetric by material composition, shape, markings, radiographic features, configuration, mechanical properties, etc. For example, it is common for guidewires to have a “J”-tip or pig tail shape at its distal end (but straight at its proximal end). Also, the distal segment of the guidewire may be relatively softer or more flexible than the proximal segment, or vice versa, the proximal segment may be relatively stiffer.

As shown here, for definition purposes, the guidewire 34 could be considered divided into four equal segments. There is a distal segment 39, which is the distal one-fourth section of the guidewire 34; there is a proximal segment 36, which is the proximal one-fourth section of the guidewire 34; and there is a middle segment in between, which are the two middle one-fourth sections 37 and 38 of the guidewire 34. This type of segmentation pattern could also be applied to the other devices or instruments of my invention.

Thus, according to my invention, the magnetic element is located somewhere on segment 36 of the guidewire 34. In some embodiments, the magnetic element is located within a distance of 5 cm from the proximal end of the guidewire 34. In some embodiments, none of the other segments (distal 39 and middle 37, 38) comprise a magnetic element.

FIG. 3 shows an example of a guidewire 50 of my invention. To specify orientation, the distal end is indicated by the reference label “D” and the proximal end is indicated by the reference label “P”. The main body 52 of the guidewire 50 is made of stainless steel. However, the proximal tip section 54 of the guidewire 50 is magnetic.

The guidewire of my invention could be used in any suitable vascular catheterization procedure. But in some embodiments, the guidewire is not a guidewire that is used in coronary angioplasty procedures, endovascular interventional radiology procedures, or other procedures in which the catheter is visualized under x-ray fluoroscopy in real-time. Because of the real-time x-ray visualization, there is less need for safeguarding against guidewire loss. Longer guidewires are typically used in such interventional procedures, whereas shorter guidewires are used in central venous catheterization procedures. As such, the guidewire of my invention could be shorter than those used in such interventional procedures. In some cases, the guidewire of my invention has a length shorter than 60 cm; and in some cases, shorter than 45 cm.

### 2. VASCULAR INSTRUMENT

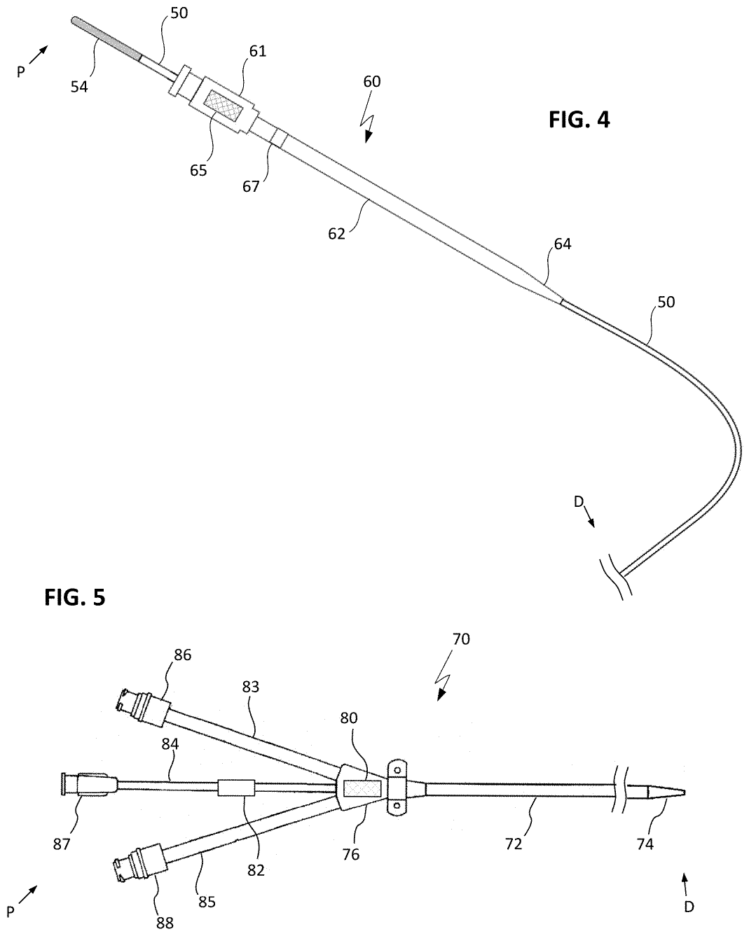

FIG. 4 shows an example of a dilation cannula of my invention used in combination with the guidewire 50 shown in FIG. 3. The dilator 60 comprises a hollow shaft 62 with a tapered tip 64 to facilitate penetration into the skin and tunnel passageway. There is a distal opening on the tapered tip 64 for receiving the guidewire 50. The guidewire 50 is inserted into the dilator 60 at its tip 64, threaded through the shaft 62, and then exits out of flange port 61. The operator firmly grasps the guidewire 50 at its proximal end and pushes the dilator 60 through the skin and subcutaneous tunnel as it tracks along the guidewire 50. The dilator 60 has a magnetic element 65 embedded in the flange port 61. If the operator loses grip on the guidewire 50 and lets it slide too far back down the dilator shaft 62 (and further into the blood vessel), then the magnetic tip 54 would magnetically couple to magnetic element 65 on the flange port 61. This magnetic coupling is sufficiently strong to impede further backsliding of the guidewire 50 down the shaft 62 and into the blood vessel. The dilator 60 also has a stainless steel cuff 67 on the shaft 62. This may be useful as backup in magnetic guidewires to prevent the guidewire 50 from backsliding even further down the dilator shaft 62.

FIG. 5 shows an example central venous catheter 70 of my invention. To specify orientation, the distal end is indicated by the reference label “D” and the proximal end is indicated by the reference label “P”. This triple-lumen catheter 70 has a main tubular shaft 72 with three separate lumens therein. One of the lumens (main lumen) opens out to opening at the distal tip 74 of the main shaft 72. The other two accessory lumens open out to side openings on the main shaft 72 (not shown here). The main lumen extends through connecting line 84, which terminates at a main port 87. During the insertion procedure, the opening at the distal tip 74 would receive a guidewire, which is already inserted inside the target vein. This could be any conventional guidewire or a magnetic guidewire of my invention. The guidewire is threaded backwards through the main shaft 72, and through main connecting line 84 until the proximal end of the guidewire exits out from main port 87. Securely grasping the proximal end of the guidewire, the catheter 70 is then advanced over the guidewire and into the target vein. Once the catheter 70 is securely in place, the entire guidewire is withdrawn out of the main port 87 and removed.

The two accessory lumens extend through connecting lines 83 and 85, which terminate at accessory ports 86 and 88, respectively. The connecting lines (83, 84, 85) are bundled together by hub 76, which assembles the separate lines together with their respective lumens in main shaft 72. The hub 76 has a magnetic strip 80 embedded therein. There is also a magnetic cuff 82 on the main connecting line 84. The magnetic strip 80 and magnetic cuff 82 would help to retain the guidewire in case of accidental release of the guidewire or inadvertent failure to withdraw the guidewire after the procedure is completed.

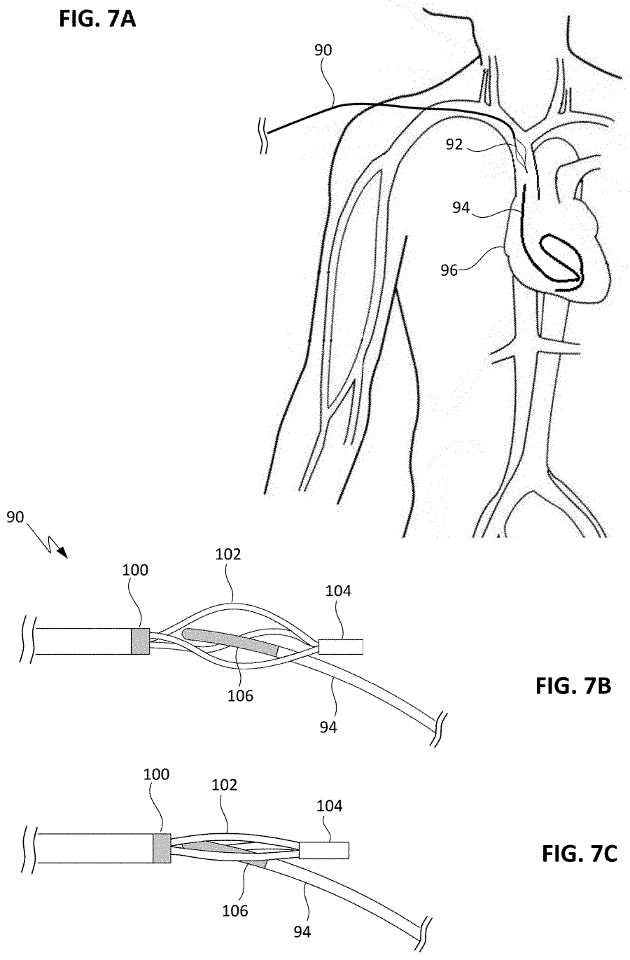

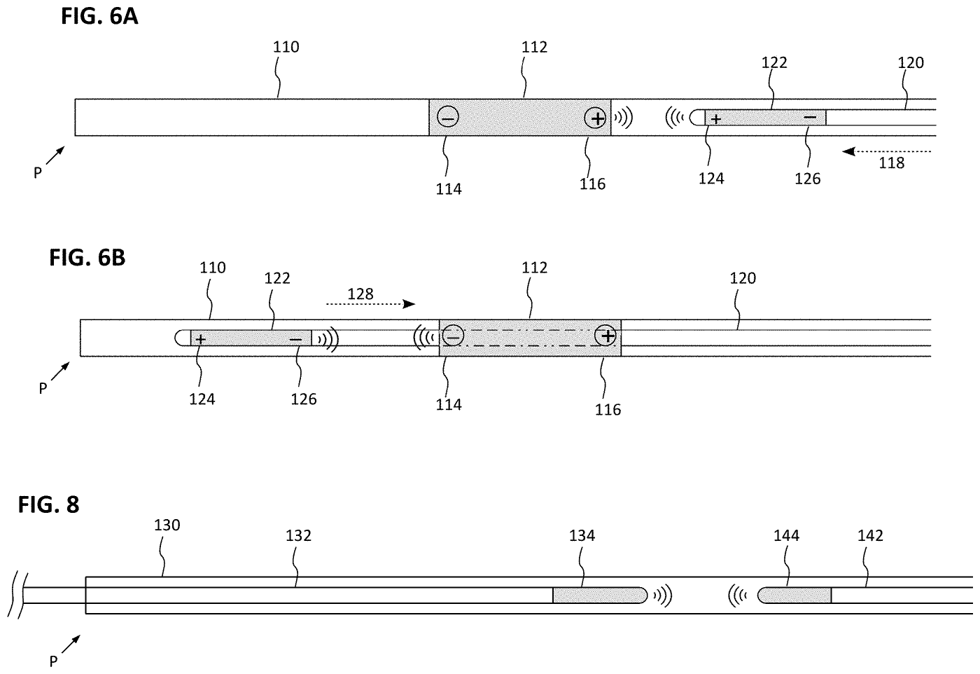

FIGS. 6A and 6B (on the landscape oriented drawing sheet alongside FIG. 8 below) show how the polarity of the magnetic elements could be arranged for enhancing function. FIG. 6A shows a catheter 110 having a magnetic segment 112, which is oriented with the magnetic north pole 114 (−) towards the proximal end and the magnetic south pole 116 (+) towards the distal end. There is a guidewire 120 having a magnetic tip 122, which is oriented with the magnetic south pole 124 (+) towards the proximal end and the magnetic north pole 126 (−) towards the distal end. As the catheter 110 is being inserted and advanced over the guidewire 120 (see-through view), the magnetic south pole 124 (+) approaches the magnetic north pole 116 (+) of the magnetic segment 112 on the catheter 110. The guidewire 120 travels in the direction of arrow 118 relative to the catheter 110. This creates some magnetic repulsion that impedes further advancement of the catheter 110 over the guidewire 120. But this resistance is easily overcome by the operator's manual manipulation to force the opposing magnetic elements to traverse each other.

FIG. 6B shows the catheter 110 and the guidewire 120 after the two magnetic elements (112, 122) have traversed each other. In this alignment, loss of grip on the guidewire 120 would be less problematic because there would be magnetic resistance to the guidewire 120 traveling backwards (in the direction of arrow 128) relative to catheter 110. The magnetic repulsion between the guidewire's magnetic north pole 126 (−) and the catheter's magnetic north pole 114 (−) would resist inadvertent traveling of the guidewire 120 in the backwards direction. Moreover, if the guidewire 120 does continue to travel in a backwards direction such that magnetic element 112 overlaps with magnetic element 122, then opposite pole attraction would cause magnetic coupling between the guidewire 120 and the catheter 110. This magnetic coupling would impede further travel of the guidewire 120 in a backwards direction.

### 3. GUIDEWIRE RETRIEVAL

The magnetic guidewire of my invention could also be useful for facilitating retrieval in the situation where the guidewire is trapped in the catheter or lost into the blood vessel. In this situation, a retrieval device could be used to retrieve the lost guidewire. The retrieval device could be another guidewire, which could be a conventional metal guidewire or a second magnetic guidewire of my invention. Also, there are various types of endovascular retrieval devices that are commonly used in interventional radiology, such as snare catheters, wire graspers, intravascular baskets, cable forceps, etc. Examples of magnetic snare catheters are described in U.S. Pat. No. 6,554,842 (Heuser et al); U.S. Pat. No. 7,144,408 (Keegan et al); and U.S. Pat. No. 5,706,827 (Ehr et al).

FIGS. 7A-7C illustrate an example of a lost guidewire situation. As shown in FIG. 7A, during a catheter insertion procedure, a guidewire 94 was inserted into the subclavian vein, but inadvertently lost. A chest x-ray showed that the lost guidewire 94 is lodged within the right ventricle 96 of the patient's heart. To retrieve the lost guidewire 94, a snare catheter 90 has been inserted through the subclavian vein and visualized under x-ray fluoroscopy. The snare catheter 90 has a set of snare hoops 92 at its distal end for snaring intravascular foreign objects.

As shown in FIG. 7B (close-up view), the snare catheter 90 has a magnetic band 100 at its distal end. Extending out from the distal end are three flexible hoops 102 that are tied together by an end cap 104. The guidewire 94 has a magnetic segment 106 at its proximal end. Magnetic attraction between the magnetic segment 106 on guidewire 94 and the magnetic band 100 on the snare catheter 90 draws in guidewire 94 close so that it becomes nestled within the hoops 102. As shown in FIG. 7C, the hoops 102 are retracted into the snare catheter 90 to cause the hoops 102 to constrict and ensnare the guidewire 94. The snare catheter 90 is then withdrawn backwards out the vein with the ensnared guidewire 94 in tow to retrieve the lost guidewire 94.

FIG. 8 illustrate an example of how two magnetic guidewires of my invention could be used in a lost guidewire situation. In this example, magnetic guidewire 142 (having a magnetic tip 144) has accidentally slid too far down the catheter 130 (see-through view). To retrieve this wayward guidewire 142, a second magnetic guidewire 132 is inserted in a backwards orientation down through catheter 130. The second guidewire 132 is advanced towards guidewire 142 until magnetic tip 134 (at the proximal end of guidewire 132) draws near magnetic tip 144 (at the proximal end of guidewire 142) so that they are magnetically coupled. The second guidewire 132 is withdrawn from catheter 130 with wayward guidewire 142 in tow behind.

### 4. CONCLUSION

The foregoing description and examples have been set forth merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A guidewire comprising:
a proximal end;
a distal end;
a magnetic element located at a proximal one-fourth segment of the guidewire.

2. The guidewire of claim 1, wherein the magnetic element is located within 5 cm from the proximal end.

3. The guidewire of claim 1, wherein the guidewire has a length of 2.5 to 600 cm long.

4. The guidewire of claim 1, wherein the guidewire has a diameter of 0.6 to 3.3 mm wide.

5. The guidewire of claim 1, wherein the guidewire has a length shorter than 60 cm.

6. The guidewire of claim 5, wherein the guidewire has a length shorter than 45 cm.

7. The guidewire of claim 1, wherein the guidewire does not have a magnetic element at a distal one-fourth segment of the guidewire.

8. The guidewire of claim 1, wherein the distal end is more flexible than the proximal end.

9. A vascular catheterization kit comprising:
(i) a guidewire comprising:
a proximal end;
a distal end;
a magnetic element located at a proximal one-fourth segment of the guidewire.
(ii) a vascular instrument comprising a magnetic element or magnetically responsive element.

10. The vascular catheterization kit of claim 9, wherein vascular instrument is a dilation cannula.

11. The vascular catheterization kit of claim 9, wherein the vascular instrument is a vascular catheter.

12. The vascular catheterization kit of claim 9, wherein the vascular instrument is a finder needle.

13. The vascular catheterization kit of claim 9, wherein the vascular instrument's magnetic element or magnetically responsive element is located at a proximal one-fourth section of the vascular instrument.

14. The vascular catheterization kit of claim 9, wherein the vascular instrument comprises a magnetic element.

15. The vascular catheterization kit of claim 9, wherein the vascular instrument comprises a magnetically responsive element.

16. The vascular catheterization kit of claim 11, wherein the vascular catheter comprises a hub and the hub comprises the magnetic element or magnetically responsive element.

17. The vascular catheterization kit of claim 14, wherein the polarity orientation of the magnetic element on the vascular instrument is opposite the polarity orientation of the magnetic element on the guidewire.

18. The vascular catheterization kit of claim 9, wherein the guidewire does not have a magnetic element at a distal one-fourth segment of the guidewire.

19. A method of retrieving a guidewire from a patient, wherein the guidewire comprises a magnetic element and is within the patient's body, the method comprising:
inserting an endovascular retrieval instrument into the patient's body;
guiding the endovascular retrieval instrument towards the guidewire;
magnetically coupling the magnetic element on the guidewire with the endovascular retrieval instrument;
withdrawing the endovascular retrieval instrument from the patient's body with the guidewire in tow.

20. The method of claim 19, further comprising ensnaring the guidewire with the endovascular retrieval instrument.
